(19) United States
(12) Patent Application Publication
(10) Pub. No.: US 2006/0190004 A1
(43) Pub. Date: Aug. 24, 2006

(54) Cutting device

(76) Inventors: Manfred Dick, Gefell (DE); Eckhard Schroeder, Eckental (DE); Martina Wilke, Erfurt (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/493,613

(22) PCT Filed: Oct. 25, 2002
(86) PCT No.: PCT/EP02/11926

§ 371 Date: Sep. 20, 2004

(30) Foreign Application Priority Data
Oct. 25, 2001 (DE) 101 52 152.9

(51) Int. Cl. A61B 18/18; A61F 11/00

(52) U.S. Cl. 606/108; 606/20

(57) ABSTRACT

A cutting device for removing an epithelium flap from an eye in a simple and safe that includes a rotation-symmetric cutting body with a cutting edge and a cavity with a bearing surface for the epithelium flap, as well as a device for fixing the epithelium flap onto the bearing surface.

The present invention relates to a cutting device for the removal of an epithelial flap from an eye and also a method for the removal of an epithelial flap from an eye.

In ophthalmology it is known to shape the cornea in the case of sight defects by ablation of tissue. Laser irradiation of an ARF excimer laser the pulsed radiation of which has a wavelength of 193 nm has proved successful for this. With radiation of this wavelength good abrasion results are achieved with very small negligible side effects. A distinction is drawn between two methods for carrying out this operation:

In the case of photorefractive keratectomy (PRK) the upper, approximately 50 micrometres-thick epithelial layer is irreversibly removed from the Bowman's membrane with various hockey knives and the laser ablation carried out on the stromal surface. Within a healing process new epithelium forms after the operation on the laser-treated surface. However the method causes pain to the patient.

In the case of laser-assisted intrastromal keratomileusis (LASIK), a stromal flap approximately 160 micrometres thick is loosened and folded away with the help of a microkeratome. The laser treatment is carried out in the intrastromal tissue and the flap folded back after the treatment. The patients suffer very little pain and their vision rapidly recovers after the operation. The use of a microkeratome is risky however and the residual thickness of the cornea available for refractive correction is less than with the PRK described above.

Recently therefore a new method has been considered in which an epithelial flap is prepared and moved aside before PRK in order to again then cover and heal the open stroma in reproducible manner. This method of laser-assisted subepithelial keratomileusis (LASEK) avoids on the one hand the surgical risk of the microkeratome and also the greater weakening of the cornea with the LASIK method shown above, but offers the patient the same low-pain healing as with LASIK (cf. “LASEK: Notes from the new fronsher”. Essay by Daniel S. Durrie, published in (?)).

However, the preparation of the epithelial flap with LASEK is currently still left to the skill of the doctor who moves the epithelium, previously partly loosened with alcohol, aside with greater or lesser success and simple surgical instruments in order to then precisely replace it. In the case of error, a LASEK thus often turns into a PRK. In addition the use of alcohol likewise leads to damage of the epithelium or else the stromal tissue if its action lasts too long and therefore also goes deeper.

The object of the present invention is therefore to prepare a method and a device that permit a simple and safe removal of an epithelial flap.

This object is achieved by a cutting device for the removal of an epithelial flap from an eye with an essentially rotation-symmetrical cutting element with a cutting edge and a free space with a bearing surface for the epithelial flap and also an apparatus for fixing the epithelial flap to the bearing surface according to claim 1 and also a method for the removal of an epithelial flap from an eye, which comprises the steps: making of an incision with a cutting device in the epithelium to approximately the depth of the Bowman's membrane, fixing of the epithelial flap to the cutting device, removal of the epithelial flap with the cutting device according to claim 12.

Particularly preferably with this cutting device an “incision” can be made in that the cutting device together with bearing surface for the epithelial flap is placed onto the eye or the epithelium and the part of the epithelium in contact with the cutting device adheres to the cutting device as epithelial flap. The epithelial flap can now be removed from the eye in that the cutting device is moved away from the eye and the epithelial flap continues to adhere to the cutting device, is therefore “torn from the eye”. Quite particularly preferably a cooled punch is applied to the epithelium as cutting device with the result that the epithelium freezes onto the punch. In a second step the punch is removed, the epithelium being removed with it. The removal of the epithelium is followed by the ablation of the tissue to correct the defective vision. Following the operation the epithelium can be reapplied, the epithelium preferably being separated from the punch by heating the punch. A therapeutic contact lens (preferably 0 dioptre refractive power) can provide protection during the healing phase. Through the choice of temperature and surface finish of the cutting device the thickness of the epithelial flap to be loosened can thus also be optimally preset. Preferred materials of the surface of the cutting device coming into contact with the epithelium are biocompatible materials.

In a development of the cutting device according to the invention the apparatus for fixing the epithelial flap includes a suction device that can produce a negative pressure between the free space and the epithelial flap. With the help of the produced negative pressure the epithelial flap is safely drawn against the bearing surface and temporarily attached to it so that the epithelial flap can be removed.

Alternatively the apparatus for fixing the epithelial flap is a cooling element. With the cooling element, the bearing surface can be cooled until the epithelial flap freezes onto it. The epithelial flap is loosened by heating the bearing surface, for example by active heat supply or by simply switching off the cooling element and heating to ambient temperature.

In a development of the cutting device the cooling element is a Peltier element. These are common elements for cooling that are correspondingly safe and cost-favourable to use. Alternatively the cooling element can be a cryogenic element.

It is advantageous if the bearing surface is rotatable vis-à-vis the cutting element. The epithelial flap temporarily attached to the bearing surface by negative pressure or freezing-on can be sheared off from the remaining epithelium by rotational movement of the bearing surface.

It is likewise advantageous if the bearing surface is radially displaceable vis-à-vis the cutting element. The shearing-off, described above, of the epithelial flap from the remaining epithelium can thus be achieved by a translational movement.

In a development of the cutting device the latter also comprises a cutting element that can separate the epithelial flap completely from the eye. The epithelial flap is separated by a cutting movement with the result that a very precise thickness of the epithelial flap is achievable.

The cutting element advantageously comprises a wire. This acts as a knife-like blade and is guided through the epithelium to be loosened by guiding the cutting wire past the cutting edge of the cutting device.

The cutting device preferably comprises a carriage, the wire being movable along the carriage. With this measure the design of the cutting device can be relatively simple.

A precise positioning of the cutting device on the eye to be treated is made possible if the cutting device comprises a suction ring for fixing the cutting device onto the eye.

In a development of the method according to the invention it is provided that the epithelial flap is completely separated from the eye by a cutting unit.

Alternatively the epithelial flap can be sheared off from the eye by rotation or displacement. Both measures named above make possible the removal of an epithelial flap of precisely defined thickness.

In a development of the method according to the invention it is provided that the epithelial flap is fixed to a contact lens. This measure offers the advantage that the removed epithelial flap can be reapplied by means of the contact lens after conclusion of the laser treatment. The contact lens makes possible a simple handling of the epithelial flap and protects the latter during the healing process.

The epithelial flap is advantageously attached to the contact lens by means of an adhesive. In this way a mechanical fixing can be avoided.

It is advantageous if the adhesive dissolves under the influence of body heat and/or lacrimal fluid. After the epithelial flap has been applied by means of the contact lens the bond thus dissolves automatically and the contact lens can be removed again.

The adhesive can advantageously be a fibrin adhesive. This is an adhesive customary in the trade which dissolves under the influence of body heat and/or lacrimal fluid.

An embodiment of the present invention is explained in more detail below using the attached drawings. There are shown in:

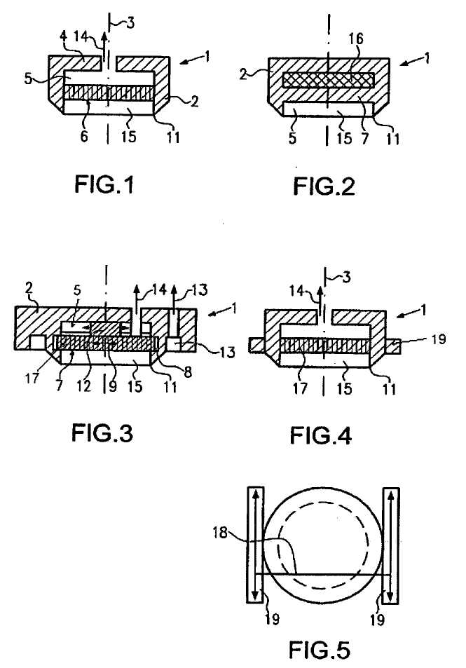

FIG. 1 a first version of a cutting device according to the invention in section;

FIG. 2 a second version of a cutting device according to the invention in section;

FIG. 3 a third version of a cutting device according to the invention in section;

FIG. 4 a fourth version of a cutting device according to the invention in side view;

FIG. 5 the fourth version according to FIG. 4 in top view.

Reference is firstly made to FIG. 1. A first version of a cutting device 1 is shown. This comprises a rotation-symmetrical cutting element 2 the axis of rotation of which is given the reference number 3 in FIG. 1. For simplicity's sake only the cutting part is shown, and not the other constituents necessary for operating and holding the cutting device 1. The cutting device 1 can be housed for example in a housing device such as a drill chuck or similar. To this end the cutting device 1 has a housing 4 that for simplicity's sake is not shown here in more detail. The cutting element 2 is cupular overall and has in the region of the housing 4 a suction device 14 with which a negative pressure can be produced in a free space 5. On the side opposite the housing 4 the cutting device 1 has a cutting edge 11. A suction ring 13 makes possible the fixing of the cutting device to the eye to be treated in that by producing a negative pressure in the suction ring 13 the whole cutting device can be suctioned to the eye.

When operating the cutting device 1 the latter is set in rotation about the axis of rotation 3. The cutting edge 11 is inserted into the epithelium up to the Bowman's membrane. By producing a negative pressure in the free space 5 with the help of the suction device 14 an epithelial flap 6 is lifted from the eye.

FIG. 2 shows a further version of a cutting device 1. In contrast to the version shown in FIG. 1, here the epithelial flap 6 is not sucked into a free space 5 by a negative pressure but frozen onto a bearing surface 7. To this end the cutting device 1 has a Peltier element or cryogenic element 16. This is arranged against the bearing surface 7 and can cool the bearing surface 7 to a temperature below the freezing point of water or a fluid to be introduced into the free space 5.

This second version of a cutting device 1 is used analogously to the version described with reference to FIG. 1. The cutting device 1 is set in rotation and the cutting edge 11 inserted into the epithelium. Once the incision is deep enough the separated epithelial flap 6 is frozen onto the bearing surface 7 by means of the Peltier or cryogenic element 16 and can thus be lifted.

A further version of a cutting device according to the invention is shown in FIG. 3. This also comprises a cutting element 2. A suction ring 13 for fixing to the eye is arranged in the outer region of the cutting element 2. An oscillating element 12 is arranged above the bearing surface 7 that bounds the free space 5. This can be a Peltier element 16 with porous or perforated baseplate 17. The bearing surface 7 is arranged radially mobile in a circular groove 8 of the cutting element 2. The oscillating element 12 rests with one side against the cutting element 2 and with another side against the bearing surface 7. If the oscillating element is operated, a translational movement of the bearing surface 7 occurs, this is indicated by a double arrow 9 in FIG. 3. The bearing surface 7 of the baseplate 17 is porous or has a plurality of through bores with the result that air can be sucked by means of the suction device 14 out of the free space 5 for the epithelial flap 6 and the epithelial flap 6 is thus sucked against the bearing surface 7.

When using the device according to FIG. 3, the procedure is as shown above, the loosening of the epithelial flap is also facilitated by a shaking motion of the bearing surface 7.

FIGS. 4 and 5 show enlarged views of the cutting device 1. An axial section is shown in FIG. 4, an outline sketch seen from above is shown in FIG. 5. The versions of the cutting device 1 described above are supplemented here by a further cutting unit 15. Here, this comprises a thin wire 18 that with the help of a carriage 19 can be guided past the cutting edge 111 as a guide for the epithelial flap 6. In this way the epithelial flap 6 is safely separated from the remaining epithelium. The epithelium can be held as in the case of the devices shown above for example by negative pressure or by freezing-on.

Alternatively the epithelial flap 6 can be held by bonding to a soft contact lens, the bonding being achieved in that in each case one component of a fibrin adhesive customary in the trade or of another substance naturally occurring in the body, optically clear and/or definedly self-dissolving under the influence of body heat or lacrimal fluid is applied to the contact lens and the other component to the epithelium, which leads to a temporary and reversible fusion of the epithelium and the fibrin adhesive and/or an adhesive substance alone is contained on the contact lens, which leads to a complete loosening of the epithelium and adhesion to the lens after marking of the epithelium surface to be loosened. The contact lens is temporarily left on the laser-treated eye for repositioning and healing of the epithelial flap.

### LIST OF REFERENCE NUMBERS

1 Cutting device
2 Cutting element
3 Axis of rotation
4 Housing
5 Free space
6 Epithelial flap
7 Bearing surface
8 Circular groove
9 Double arrow
11 Cutting edge
12 Oscillating element
13 Suction ring
14 Suction device
15 Cutting unit
16 Peltier or cryogenic element
17 Baseplate
18 Wire
19 Carriage

1-18. (canceled)

19. A cutting device for removing an epithelial flap from an eye, comprising:
an essentially rotation-symmetrical cutting element having a cutting edge and a free space;
a bearing surface for the epithelial flap; and
a fixing device configured to fix the epithelial flap to the bearing surface.

20. The cutting device as recited in claim 19, wherein the fixing device includes a suction device configured to produce a negative pressure between the free space and the epithelial flap.

21. The cutting device as recited in claim 19, wherein the fixing device includes a cooling element.

22. The cutting device as recited in claim 21, wherein the cooling element includes a Peltier element.

23. The cutting device as recited in claim 21, wherein the cooling element includes a cryogenic element.

24. The cutting device as recited in claim 19, wherein the bearing surface is rotatable relative to the cutting element.

25. The cutting device as recited in claim 19, wherein the bearing surface is radially displaceable relative to the cutting element.

26. The cutting device as recited in claim 19, further comprising a cutting unit configured to separate the epithelial flap completely from the eye.

27. The cutting device as recited in claim 26, wherein the cutting unit includes a wire.

28. The cutting device as recited in claim 27, wherein the cutting unit includes a carriage, the wire being movable along the carriage.

29. The cutting device as recited in claim 19, further comprising a suction ring configured to fix the cutting device onto the eye.

30. A method for removing of an epithelial flap from an eye, the method comprising:
making of an incision in the epithelium to approximately a depth of a Bowman's membrane of the eye using a cutting device;
fixing the epithelial flap to the cutting device; and
removing the epithelial flap using the cutting device.

31. The method as recited in claim 30, further comprising completely separating the epithelial flap from the eye using a cutting unit prior to the removing.

32. The method as recited in claim 30, further comprising shearing the epithelial flap off from the eye using at least one of a rotation or displacement of the epithelial flap.

33. The method as recited in claim 30, further comprising attaching the epithelial flap to a contact lens.

34. The method as recited in claim 33, wherein attaching is performed using an adhesive.

35. The method as recited in claim 34, wherein the adhesive is dissolvable under an influence of at least one of a body heat and a lacrimal fluid.

36. The method as recited in claim 34, wherein the adhesive includes a fibrin adhesive.
